(19) United States
(12) Patent Application Publication
(10) Pub. No.: US 2023/0114196 A1
(43) Pub. Date: Apr. 13, 2023

(54) HEARING PROTECTOR SYSTEMS

(71) Applicant: 3M INNOVATIVE PROPERTIES COMPANY, St. Paul, MN (US)

(72) Inventors: Lars O. Reibner, VÄRNAMO (SE); Ken G.M. Johansson, BOR (SE); Patric A. Anvegård, Bredaryd (SE)

(21) Appl. No.: 17/904,827

(22) PCT Filed: Feb. 17, 2021
(86) PCT No.: PCT/IB2021/051341

§ 371 Date: Aug. 23, 2022

Related U.S. Application Data
(60) Provisional application No. 62/981,057, filed on Feb. 25, 2020.

(51) Int. Cl. H04W 4/10; H04M 1/60; H04R 1/10; A61F 11/14; A61F 11/12

(52) U.S. Cl. CPC H04W 4/10; H04M 1/6066; H04R 1/10; A61F 11/145; A61F 11/12

(57) ABSTRACT

A hearing protection device is presented. The hearing protection device includes a first hearing protection unit and a second hearing protection unit. Each of the first and second hearing protection units include a microphone that receives ambient sound, a processor that provides level dependent attenuation of the ambient sound, and a speaker that broadcasts the sound into a user's ear. The hearing protection device also includes a first communication unit, which broadcasts a digital push-to-talk signal over a first communication protocol. The first communication protocol is a low energy communication protocol. The hearing protection device also includes a second communication unit, which broadcasts a digital communication signal over a second communication protocol. The hearing protection device also includes a processor that facilitates pairing of the hearing protection device with a mobile device that receives the push-to-talk signal and, based on the push-to-talk signal, receives the digital communication signal and transmits the digital communication signal over a cellular network.

### BACKGROUND

The “push to talk” functionality available on some mobile phone systems operate similarly to that of a two-way radio or walkie-talkie. The push to talk function allows one party to quickly and directly poll another party's mobile telephone through a dedicated channel. push to talk functionality was originally offered through Motorola's Integrated Digital Enhanced Network (iDEN™) and Nextel Communication's Direct Connect™ service, and has now been extended to other networks, including Code Division Multiple Access (CDMA) networks and Global System for Mobile communications (GSM) networks.

### SUMMARY

A hearing protection device is presented. The hearing protection device includes a first hearing protection unit and a second hearing protection unit. Each of the first and second hearing protection units include a microphone that receives ambient sound, a processor that provides level dependent attenuation of the ambient sound, and a speaker that broadcasts the sound into a user's ear. The hearing protection device also includes a first communication unit, which broadcasts a digital push-to-talk signal over a first communication protocol. The first communication protocol is a low energy communication protocol. The hearing protection device also includes a second communication unit, which broadcasts a digital communication signal over a second communication protocol. The hearing protection device also includes a processor that facilitates pairing of the hearing protection device with a mobile device that receives the push-to-talk signal and, based on the push-to-talk signal, receives the digital communication signal and transmits the digital communication signal over a cellular network.

The details of one or more examples of the disclosure are set forth in the accompanying drawings and the description below. Other features, objects, and advantages of the disclosure will be apparent from the description and drawings, and from the claims.

### BRIEF DESCRIPTION OF THE DRAWINGS

FIG. 1 illustrates a personal protective device in which embodiments of the present invention are useful.

FIG. 2 illustrates a plurality of personal protective devices in a communication network in which embodiments of the present invention are useful.

FIG. 3 illustrates components of a Bluetooth® enabled personal protective device in accordance with embodiments of the present invention.

FIG. 4 illustrates a component block diagram of push-to-talk over cellular enabled personal protective equipment in accordance with embodiments of the present invention.

FIG. 5 illustrates a method of using push-to-talk over cellular enabled personal protective equipment. FIGS. 6-8 illustrate example devices that can be used in the embodiments shown in previous Figures.

Repeated use of reference characters in the specification and drawings is intended to represent the same or analogous features or elements of the disclosure. It should be understood that numerous other modifications and embodiments can be devised by those skilled in the art, which fall within the scope and spirit of the principles of the disclosure. The figures may not be drawn to scale.

### DETAILED DESCRIPTION

Noisy environments, such as worksites, airfields, and the like, may include noise sources that have the potential to damage the hearing of a person. A person operating in a noisy environment may experience hearing loss from acute acoustic trauma or gradually developing noise-induced hearing loss. Acute acoustic trauma may cause hearing loss from a one-time exposure to an excessive noise level, whereas gradually developing noise-induced hearing loss may result from exposure to unsafe noise levels over an extended period of time.

To prevent hearing damage, a person operating in noisy environment may wear hearing protection. Active hearing protection may include earmuffs that permit certain types of noise to pass through to a person by electronically reducing decibels or filtering out frequencies.

Although various forms of hearing protection may provide adequate protection against excessive noise, persons wearing such hearing protection may need to communicate with one another. In noisy environments, communicating with one another may be difficult while wearing hearing protection due to noise from noise sources and filtering from the hearing protection. In some scenarios, a person wearing hearing protection may carry an additional and separate communication device to facilitate communication with other individuals in a noisy environment. Although separate communication devices may facilitate such communication, the person may be required to carry two separate devices (hearing protection and communication device), which may be coupled by a cable. Such two-device configurations can impede a person's movement, hinder the likelihood of communicating, and/or potentially introduce safety risks (e.g., if the cable becomes entangled on the user or other objects in the environment).

Additionally, specialized communication devices require specialized maintenance and user training to become accustomed to the user interface and technical functionality of the specialized communication unit. While a communication unit provides the functionality of push-to-talk technology, a more flexible configuration is desired. The specialized communication units would receive a push-to-talk command from the headset. When the user pushes the button on the headset and a floor is available, the PTT over cellular channel opens one way from this user broadcasting his audio to all other connected users.

Workers in noisy environments are usually carrying cellular phones, either personal or business issued. It is desired to have a push-to-talk over cellular implementation in active hearing protection devices. As described herein, in some embodiments, worker cellular phones can have a mobile application installed that will provide push-to-talk over cellular functionality for personal protective hearing devices. A user can receive incoming audio and, by activating a push-to-talk feature, can also send communication using the cellular network of their cellular phones. Using the cellular phone allows for a user to mimic classic two-way radio technology, with higher audio quality than offered by analog communication radios. Using a mobile cellular phone also provides a user with a familiar, simpler interface. The mobile cellular phone is easier to replace than a traditional communications unit, is compatible with more devices, and can be used anywhere in the world. Recipients in the conference can be anywhere in the world. This is compared to traditional PTT where the locations of all participants are limited to a certain geographical radius depending on the radio range of the devices, which may be extended by repeater networks.

“Active hearing protection,” as used herein, includes one or more microphones that receive ambient sound from a user's surroundings and uses one or more speakers to play it back at a safe decibel level. Active hearing protection devices use electronic circuitry to pick up ambient sound through the microphone and convert them to safe levels before playing it back to the user through a speaker. Additionally, active hearing protection may comprise filtering out undesired sound content, for example actively reducing the sound of a gunshot while providing human speech at substantially unchanged levels. However, while some embodiments of the present invention expressly contemplate units with active hearing protection, it is expressly contemplated that passive hearing protection units are included in other embodiments.

First, a sound signal is received by a microphone in an active hearing protection unit. The received sound signal is converted to an electronic signal for processing. After processing the sound signal such that all frequencies are at safe levels, the sound signal is reproduced and played back to a user through a speaker.

Some active hearing protection units are level dependent, such that an electronic circuit adapts the sound pressure level. Level dependent hearing protection units help to filter out impulse noises, such as gunshots from surrounding noises, and/or continuously adapt all ambient sound received to an appropriate level before it is reproduced to a user. Active hearing protection units, specifically level dependent active hearing protection units, may be necessary to facilitate communication in noisy environments, or environments where noise levels can vary significantly, or where high impulse sounds may cause hearing damage. A user may need to hear nearby ambient sounds, such as machine sounds or speech, while also being protected from harmful noise levels.

Hearing protection units with active hearing protection can also receive digital signals from a source and provide them through the speakers inside the active hearing protection unit. For example, existing Bluetooth®-enabled headsets can stream audio, music, and play audio from a cell phone call. Existing headsets with push-to-talk technology can also, with the push of a button by the operator, send out a push-to-talk command to a mobile communications unit.

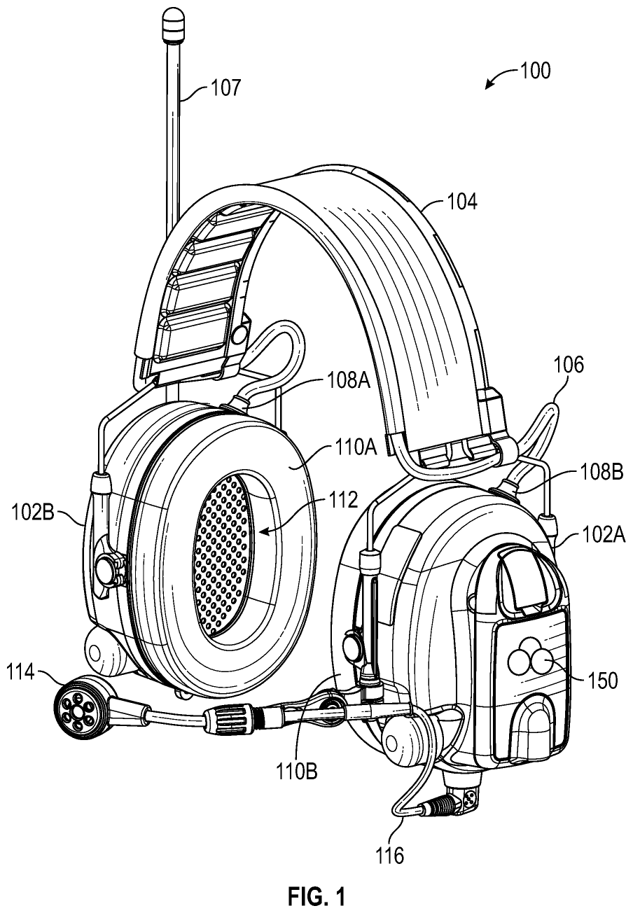

FIG. 1 illustrates a personal protective device in which embodiments of the present invention are useful. As described herein, personal protection device 100 is enabled with Bluetooth® and Bluetooth® SMART® (also referred to as Blueooth® low energy, or “BLE” as referred to here) technology.

This disclosure is directed to an acoustic headset for providing hearing protection, wherein the acoustic headset includes both digital and analog components for transmission and receipt of digital and analog wireless audio communications. Acoustic headset 100 may provide for both digital and analog two-way communications using components that are integrated within auditory cups of the acoustic headset. As such, no additional digital components external to the acoustic headset may be used to assist a digital component of headset 100 in the transmission and receipt of the digital wireless audio communications from a mobile cellular device as described herein. Additionally, no additional analog components external to the acoustic headset may be used to assist an analog component of headset 100 in the transmission and receipt of the analog wireless audio communications.

FIG. 1 is a diagram illustrating an example protective headset 100 that includes digital and analog two-way communication components, including Bluetooth® communication functionality. As shown in FIG. 1, protective headset 100 includes first auditory cup 102A and second auditory cup 102B. Auditory cups 102A and 102B are physically coupled by a stirrup or headband 104. Stirrup or headband 104 may be comprised of any rigid or semi-rigid material, such as plastic, aluminum, steel or any other suitable material. However, it is expressly contemplated that a headset 100 may not include a stirrup or headband. For example, a behind-the-neck headset 100 may have coupled auditory cups 102A and 102B connected behind a user's neck, instead of over a user's head. Additionally, it is expressly contemplated that a helmet version of headset 100 may also be practiced, such that auditory cups 102A and 102B are incorporated into a helmet.

Protective headset 100 may include one or more antennas, such as antenna 107 to receive digital and/or analog signals from devices that are remote from protective headset 100. As further described in this disclosure, auditory cups 102A and 102B may include hardware that provides digital and analog two-way communication. Such hardware may be implemented, in part, with printed circuit boards and components integrated in the printed circuit boards.

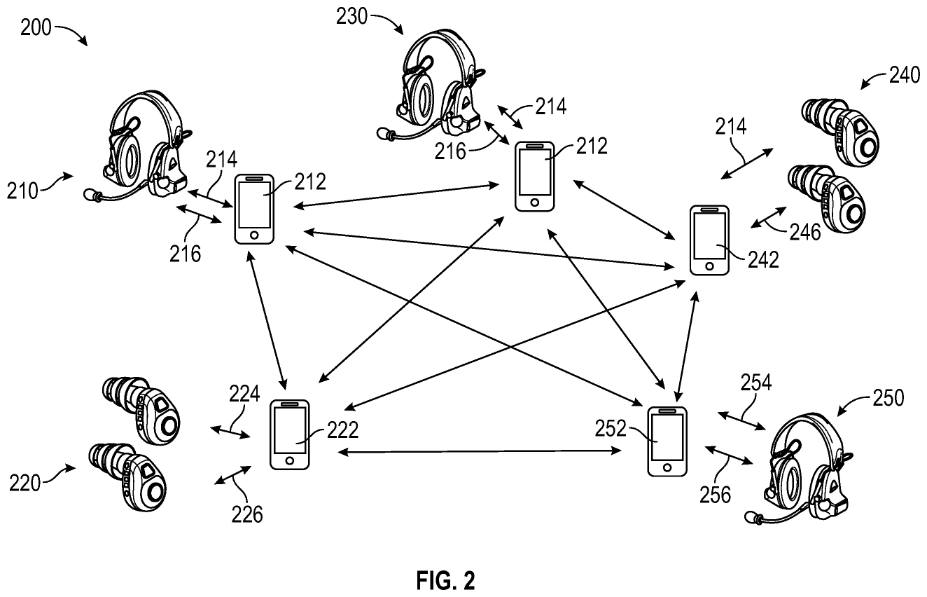

In examples where the hardware that provides digital and analog two-way communication is distributed between first auditory cup 102A and second auditory cup 102B, such as FIG. 1, one or more communication links may communicatively couple the distributed hardware. For instance, headset 100 includes communication link 106, which communicatively and physically couples hardware auditory cups 102A, 102B that provide digital and analog two-way communication. Examples of communication link 106 may include one or more strands of wire comprised of copper, aluminum, silver or other suitable conducting material. The ends of communication link 106 may enter auditory cups 102A, 102B via ports 108A, 108B, and couple to hardware such as printed circuit boards included within each of auditory cups 102A, 102B. Similarly, while a wired connection between auditory cups 102A, 102B is illustrated and described, it is expressly contemplated that a wireless connection may also be possible, particularly for embodiments where active hearing protection is provided through earbuds, such as hearing protection device 220 of FIG. 2. FIG. 1 illustrates cushions 110A and 110B that are attached or otherwise affixed to auditory cups 102A and 102B. Cushions 110A and 110B may abut around the ears of the wearer of headset 100. Cushions 110A and 110B contribute in the capability of auditory cups 102A, 102B to dampen or otherwise reduce ambient sound from an environment outside of auditory cups 102A, 102B. Cushions 110A and 110B may be comprised of any compressible and/or expanding material, such as foam, gel, air, or any other such suitable material. Auditory cups 102A, 102B may be comprised of any rigid or semi-rigid material, such as a plastic, which in some cases, may be a non-conductive, dielectric plastic.

Auditory cup 102B includes loudspeaker component 112. In some examples, auditory cup 102A may also include a loudspeaker component similar to or the same in structure in functionality as loudspeaker component 112. Loudspeaker component 112 may emit sound based on an analog or digital signal received or generated by headset 100. Loudspeaker component 112 may include one or more electroacoustic transducers that convert electrical audio signals into sound. Some example loudspeaker components may include a magnet, voice coil, suspension and diaphragm or membrane. Loudspeaker component 112 may be communicatively coupled to the hardware that provides digital and analog two-way communication. As an example, if headset receives a signal representing voice communication, loudspeaker component 112 may emit sound corresponding to the signal.

Headset 100 also includes a microphone 114. Microphone 114 may be communicatively and/or physically coupled to the hardware that provides digital and analog two-way communication is distributed between first auditory cup 102A and second auditory cup 102B, such as FIG. 1. While microphone 114 is illustrated as extending from a cup 102A, it could also be placed on an exterior of either auditory cup 102A, 102B. Microphone 114 may be any device that translates sound into electrical audio signals. For instance, a person wearing headset 100 may speak, thereby generating sound that is received by microphone 114. Microphone 114 may convert the spoken sounds into electrical audio signals that are received by the hardware that provides digital and analog two-way communication. Electrical audio signals may be transmitted to hardware that provides digital and analog two-way communication via communication link 116.

Push-to-talk functionality 150 is illustrated in FIG. 1 as being present on one earmuff. However, other configurations are also contemplated, such as the other earmuff, on the headband 104, on the headband 104, in both earmuffs 102A and 102B, or another suitable or another suitable position. Push-to-talk functionality 150 may include both an initiate and/or end button, as well as volume control, for example. In some embodiments, a user must maintain a push-to-talk button in an activated position to maintain an ability to transmit. In other embodiments, push-to-talk functionality is operational until a user deactivates it. While FIG. 1 illustrates a mechanical button that a user presses to activate or de-active, it is expressly contemplated that it could also be a touch-activated sensor, or another mechanical functionality.

In some embodiments, the push-to-talk functionality 150 includes a voice-operated switch (VOX), which operates to turn on the microphone when sound over a certain threshold is detected, enabling the microphone to activate when a speaker is detected, and deactivate when a speaker stops speaking.

A user wearing headset 100 may, for example, press, or otherwise activate, the PTT (Push-to-Talk) 150 functionality. The PTT 150 functionality is integrated in the hearing protector and, using a digital communication component connects to a mobile cellular phone to establish a connection. Using an installed application on the mobile cellular phone, the cellular phone then can establish communication with a second user's mobile cellular phone, which can then broadcast communication to the second user's headset. In some embodiments, the cellular phone establishes communication with many other cellular phones simultaneously such that a message can be broadcast to multiple listeners.

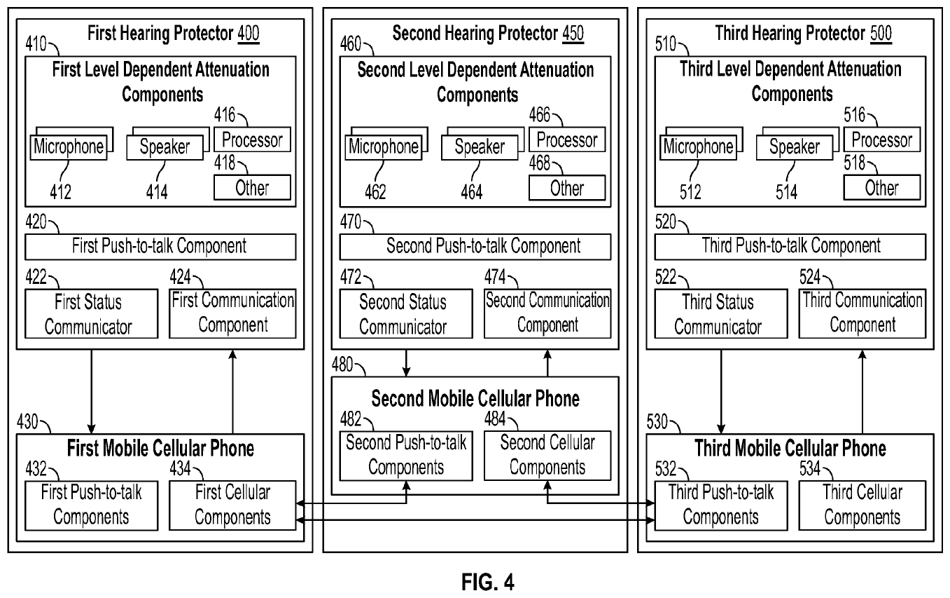

The PTT 150 functionality includes an active signaling component that, upon being engaged, sends a signal to the user's mobile cellular phone, the signal indicative that a user wants to actively communicate using the PTT-over-cellular communication. Thus, as discussed in greater detail in FIG. 4, headset 100 is configured to both passively receive digital communication signals and actively send digital signals to initiate communication.

FIG. 2 illustrates a plurality of personal protective devices in a communication network 200 in which embodiments of the present invention are useful. Devices 210, 230 and 250 are over-the-ear protective hearing headsets, as illustrated in FIG. 1 in detail.

Devices 220 and 240 are in-ear protective hearing devices. Both types of active hearing protection may have PTT-over-cellular functionality. While five devices are illustrated in FIG. 2, this is by example only. More, or fewer devices could also be present in other embodiments, such as only two devices, three devices, four devices, or up to 10, up to 20 or more than 20 devices.

Devices 210, 230 and 250 are active hearing protection device that receives ambient sounds and provides appropriate sound compression or attenuation of the ambient sound before broadcasting it through speakers inside each earmuff. Device 210 may also have at least some sound localization features, such that sounds are presented to a user indicative of where they are in relation to the user, with sounds on the user's left being reproduced louder on the left side. Additionally, device 210 may provide other functionality that is not described in detail herein, including other passive sound broadcasting, including FM/AM/DAB radio, digital two-way radio such as DMR or DPMR, and may also allow for data exchange over WiFi as well as other wired inputs.

Device 210 is illustrated as an over-the-ear hearing protection device. Device 210 is paired with a mobile cellular device 212, for example using Bluetooth®, BLE, or another suitable pairing protocol. Two different communication protocols are present between device 210 and device 212. First communication protocol 214 is a low energy protocol that transmits a PTT signal from device 210 to device 212. An indication of an open floor may be sent back, from device 212 to 210. The indication may be auditory, visual, haptic or other suitable feedback. When a PTT floor is open, an audio signal may be transmitted from device 210 to device 212 using second communication protocol 216. Protocol 216 may be any suitable digital communication protocol such as Bluetooth®, BLE, or another protocol. Lower energy requirements of the audio signal transmission are considered, as well as quality requirements, when selecting second protocol 216. An incoming PTT signal from another device, such as device 220, may also be received by device 212 and sent to device 210 through protocol 216.

Device 220 is illustrated as an in-ear hearing protection device. Device 220 is paired with a mobile cellular device 222, for example using Bluetooth®, BLE, or another suitable pairing protocol. Two different communication protocols are present between device 220 and device 222. First communication protocol 224 is a low energy protocol that transmits a PTT signal from device 220 to device 222. An indication of an open floor may be sent back, from device 222 to 220. The indication may be auditory, visual, haptic or other suitable feedback. When a PTT floor is open, an audio signal may be transmitted from device 220 to device 222 using second communication protocol 226. Protocol 226 may be any suitable digital communication protocol such as Bluetooth®, BLE, or another protocol. Lower energy requirements of the audio signal transmission are considered, as well as quality requirements, when selecting second protocol 226. An incoming PTT signal from another device, such as device 210, may also be received by device 222 and sent to device 220 through protocol 226.

Device 230 is illustrated as an over-the-ear hearing protection device. Device 230 is paired with a mobile cellular device 232, for example using Bluetooth®, BLE, or another suitable pairing protocol. Two different communication protocols are present between device 230 and device 232. First communication protocol 234 is a low energy protocol that transmits a PTT signal from device 230 to device 232. An indication of an open floor may be sent back, from device 232 to 230. The indication may be auditory, visual, haptic or other suitable feedback. When a PTT floor is open, an audio signal may be transmitted from device 230 to device 232 using second communication protocol 236. Protocol 236 may be any suitable digital communication protocol such as Bluetooth®, BLE, or another protocol. Lower energy requirements of the audio signal transmission are considered, as well as quality requirements, when selecting second protocol 236. An incoming PTT signal from another device, such as device 220, may also be received by device 232 and sent to device 230 through protocol 236.

Device 240 is illustrated as an over-the-ear hearing protection device. Device 240 is paired with a mobile cellular device 242, for example using Bluetooth®, BLE, or another suitable pairing protocol. Two different communication protocols are present between device 240 and device 242. First communication protocol 244 is a low energy protocol that transmits a PTT signal from device 240 to device 242. An indication of an open floor may be sent back, from device 242 to 240. The indication may be auditory, visual, haptic or other suitable feedback. When a PTT floor is open, an audio signal may be transmitted from device 240 to device 242 using second communication protocol 246. Protocol 246 may be any suitable digital communication protocol such as Bluetooth®, BLE, or another protocol. Lower energy requirements of the audio signal transmission are considered, as well as quality requirements, when selecting second protocol 246. An incoming PTT signal from another device, such as device 220, may also be received by device 242 and sent to device 240 through protocol 246.

Device 250 is illustrated as an over-the-ear hearing protection device. Device 250 is paired with a mobile cellular device 252, for example using Bluetooth®, BLE, or another suitable pairing protocol. Two different communication protocols are present between device 250 and device 252. First communication protocol 254 is a low energy protocol that transmits a PTT signal from device 250 to device 252. An indication of an open floor may be sent back, from device 252 to 250. The indication may be auditory, visual, haptic or other suitable feedback. When a PTT floor is open, an audio signal may be transmitted from device 250 to device 252 using second communication protocol 256. Protocol 256 may be any suitable digital communication protocol such as Bluetooth®, BLE, or another protocol. Lower energy requirements of the audio signal transmission are considered, as well as quality requirements, when selecting second protocol 256. An incoming PTT signal from another device, such as device 220, may also be received by device 252 and sent to device 250 through protocol 256.

It is also expressly contemplated that the hearing protection device may be a dual hearing protection system, such as that disclosed in U.S. Provisional Patent Application 62/909989, filed on Oct. 3, 2019, the contents of which are hereby incorporated by reference.

In one embodiment, when using a push to talk feature, a user may first select a recipient from a directory, then presses a push to talk button. For example, the user may select a recipient using an interface on the mobile cellular phone. However, in another embodiment, activating a push-to-talk over cellular feature allows the user to communicate over a network to multiple users, similar to the functionality of a plurality of walkie-talkies. In some embodiments, the push-to-talk functionality includes a voice-operated switch (VOX), which operates to turn on the microphone when sound over a certain threshold is detected, enabling the microphone to activate when a speaker is detected, and deactivate when a speaker stops speaking.

One of the hearing protection devices, 210, 220, 230, 240 or 250, using a first digital communication protocol, transmits a request to a mobile cellular device (not shown). Provided the floor is available for communication, the mobile cellular device will receive an audio transmission from the hearing protection devices, using a second digital communication protocol, and transmit the audio transmission using a third communication protocol. In some embodiments, the first and second communication protocols correspond to a Bluetooth® or a BLE protocol. In some embodiments, the third communication protocol is either a Bluetooth® protocol or a cellular network protocol, such as 2G, 3G, 4G, 5G, CDMA, TDMA, FDMA or any other suitable data provider that is compatible with a given mobile cellular device.

Conventional push to talk systems require that when using the push to talk function, a user must continuously hold down the push to talk button while speaking. The user must then release the push to talk button to release the floor and receive messages from others.

It will be appreciated that embodiments of the invention described herein may be comprised of one or more conventional processors and unique stored program instructions that control the one or more processors to implement, in conjunction with certain non-processor circuits, some, most, or all of the functions of establishing push to talk communication sessions as described herein. The non-processor circuits may include, but are not limited to, a radio receiver, a radio transmitter, signal drivers, clock circuits, power source circuits, and user input devices. As such, these functions may be interpreted as steps of a method to perform establishment of push to talk communication sessions. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation. Systems and methods for establishing a push to talk communication session for a protective hearing device is described.

Several different digital communication protocols may be useful in embodiments herein. Classic Bluetooth® relies on the 2.4 GHz radio frequency to transmit data over a wireless link to another device within a range up to about 10 meters.

The Bluetooth® Low Energy (BLE) protocol is another protocol that may be used herein for transmitting either a PTT-over-cellular signal from a headset to a mobile device, or to transmit an audio signal from the headset to the mobile device. BLE is not backward-compatible with the classic Bluetooth® Basic Rate/Enhanced Data Rate (BR/EDR) protocol. The Bluetooth® 4.0 specification permits devices to implement either or both of the BLE and BR/EDR systems.

Bluetooth® Low Energy uses the same 2.4 GHz radio frequencies as classic Bluetooth®, which allows dual-mode devices to share a single radio antenna. BLE does, however, use a simpler modulation system. Mesh specification enables using Bluetooth® Low Energy for many-to-many device communications for home automation, sensor networks and other applications.

Energy consumption is an important parameter for active hearing protection units because the units are battery-powered and wireless. The battery must fit within the active hearing protection unit, not add significant weight or a weight imbalance between the two earmuffs and must provide power to the level dependent processing functions, as well as any communication functions, for a useful amount of time in between charges. The advancement of low energy communication protocols has enabled additional communication functionality in more user-friendly configurations than previously available.

The majority of current low energy application profiles are based on the Generic Attribute Profile (GATT), a general specification for sending and receiving short pieces of data, known as attributes, over a low energy link. The Bluetooth® mesh profile is an exception to this rule, being based on the General Access Profile (GAP). Other low energy protocols may also be suitable, such as HOGP (HID (Human Interface Device Protocol) over GAP Protocol).

Bluetooth® Low Energy technology operates in the same spectrum range (the 2.400-2.4835 GHz ISM band) as classic Bluetooth® technology but uses a different set of channels. Instead of the classic Bluetooth® 79 1-MHz channels, Bluetooth® Low Energy has 40 2-MHz channels. Within a channel, data is transmitted using Gaussian frequency shift modulation. The bit rate is 1 Mbit/s (with an option of 2 Mbit/s in Bluetooth® 5), and the maximum transmit power is 10 mW (100 mW in Bluetooth® 5). Further details are given in Volume 6 Part A (Physical Layer Specification) of the Bluetooth® Core Specification V4.0.

Bluetooth® Low Energy uses frequency hopping to counteract narrowband interference problems. Classic Bluetooth® also uses frequency hopping but the details are different; as a result, while both FCC and ETSI classify Bluetooth technology as an FHSS scheme, Bluetooth® Low Energy is classified as a system using digital modulation techniques or a direct sequence spread spectrum.

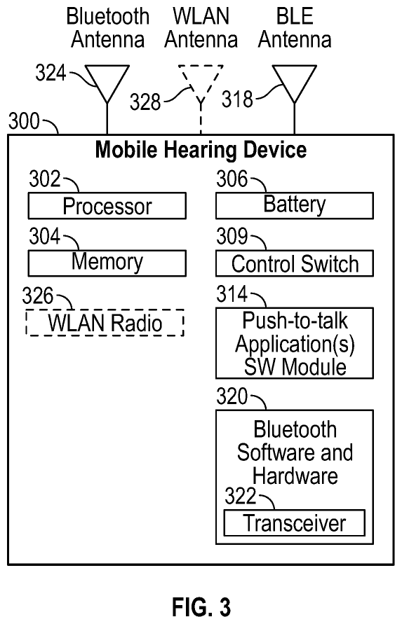

FIG. 3 illustrates a block diagram of a device 300 according to one embodiment of the invention. The device 300 can be an over-the-ear protective hearing device, an in-ear protective hearing device, and/or any other personal protective device that includes PTT-over-cellular functionality. Device 300 includes a processor 302. The processor 302 can be a genera-purpose microprocessor or a customizable integrated circuit such as an application-specific integrated circuit (ASIC). In one embodiment, the processor includes a digital signal processor (DSP).

Device 300 can also include memory 304. The memory 304 can include random access memory (RAM), read-only memory (ROM), flash memory, or any other suitable memory. The memory 304 can reside on an erasable programmable read only memory (EPROM). A battery 306 is present within device 300 to provide power. Battery 306 can be a disposable battery or a rechargeable battery. Any suitable battery can be used.

The wireless communication device 300 also includes operating system software that can reside in the memory 304. A push to talk software application 314 can also reside in the memory 304. The push to talk software application 314 can interface with the operating system software.

In another embodiment, the push to talk software application 314 enables the wireless device 300 to perform push to talk functionality without an operating system software interface. Device 300 includes a physical control switch 309 that enables a push to talk session with device 300. The control switch 309 can be a push button, for example.

The wireless communication device 300 can optionally include a BLE module coupled to an antenna 318 that allows the wireless communication device 300 to communicate with a mobile cellular device using the BLE protocol described above. Device 300 can optionally include a wireless local area network (WLAN) radio 326 and antenna 328 to allow the wireless device 200 to communicate over a wireless local area network.

Device 300 may also include a Bluetooth® radio module 320. The Bluetooth® radio module 320 includes both hardware and software. For example, the Bluetooth® module 320 can include a wireless transceiver 322 as well as software. The software can include Bluetooth® radio drivers and Bluetooth® application software. The wireless transceiver 322 is coupled to a Bluetooth® antenna 324. The wireless transceiver 322 and

Bluetooth® antenna 324 are configured to transmit and receive audio and data signals over relatively short distances. For example, Device 300 can transmit an audio signal through to a mobile device using a Bluetooth® communication protocol when device 300 is in range of the mobile cellular device and when a PTT-over-cellular functionality is active. The communication is achieved when the devices are “paired” with each other.

In operation, device 300 is configured to communicate wirelessly with other devices over a public or private communication network. In one mode of operation, device 300 includes push to talk functionality through a mobile cellular phone as described herein. Push to talk functionality generally allows one party to speak to many parties in a “walkie-talkie”-like mode. In other embodiments, a push to talk session can occur between only two parties, using paired mobile cellular devices as intermediaries.

In order to initiate a push to talk session, a user wishing to transmit audio signal activates the control switch 309. The activation of the control switch 309 indicates that the user wishes to request the floor. The user will gain the floor if the floor is available. Many PTT systems use a first-in, first-out system to determine the order with which the floor will be gained. The floor is release when the user releases the control switch 309. In other embodiments, the control switch 309 is a momentary switch and the user must activate the control switch once to gain the floor and once to release the floor. According to one embodiment of the invention, the wireless communication device 300 is paired with a mobile cellular device through BLE communication protocols, and the mobile cellular device, upon receiving a request to gain the floor, checks the floor for availability and, if available, receives an audio signal from device 300 using Bluetooth® or Bluetooth® low energy protocols, or another suitable low energy protocol. For example, pairing can be achieved through standard Bluetooth pairing. Alternatively, the pairing can be achieved using fast pairing, through attempting to use common PIN codes.

The call button 302 on the device 300 is typically used to send commands to a mobile cellular device. In one embodiment of the invention, the commands sent from the call button 302 are monitored through software on the device 300. When the appropriate command is sent, the software diverts the command to the push to talk module 314 on the wireless communication device 300. The push to talk module 314 then initiates a push to talk session by requesting the floor. However, while FIG. 3 illustrates an embodiment where push-to-talk application module 314 and associated memory 304 are part of a personal protection device 300, it is expressly contemplated that these components may be stored on the mobile cellular device instead, in order to conserve battery power of battery 306 and increase use time in between charges. PTT over Bluetooth® technology is discussed in more detail in U.S. 2011/0143664 to Fuccello, published Jun. 16, 2011.

For example, the Bluetooth® transceiver may be a local area, low power transmitter capable of sending electronic signals including voice and software control commands to, for example, a wireless communication device. Thus, the Bluetooth®-enabled wireless device may include support circuitry coupled with the call button for detecting an actuation of the call button. The support circuitry can include a microprocessor, memory, signal processing and other logic circuitry. An audio codec can be used to convert audible voice received by a microphone into logic signals for transmission to a remote device. The audio codec is coupled to the Bluetooth® transceiver. The Bluetooth® transceiver is coupled to an antenna.

FIG. 4 illustrates a component block diagram of push-to-talk over cellular enabled personal protective equipment in accordance with embodiments of the present invention. In some embodiments, the push-to-talk functionality includes a voice-operated switch (VOX), which operates to turn on the microphone when sound over a certain threshold is detected, enabling the microphone to activate when a speaker is detected, and deactivate when a speaker stops speaking. As illustrated in FIG. 4, three hearing protection devices 400, 450 and 500 may have need to communicate with each other. For example, wearers of devices 400, 450, 500 may be in the same worksite, or may include workers communicating with an offsite supervisor. Other use cases are also envisioned.

Each of devices 400, 450 and 500 are paired with a mobile cellular device 430, 480, 530, respectively. As illustrated in FIG. 4, devices 400, 450 and 500 do not communicate directly with one another, but can communicate by using the cellular network, or other communication protocol, of mobile cellular devices 430, 480 and 530, respectively.

First hearing protection device 400 includes first level dependent attenuation components 410, including microphones 412 that detect ambient sound, a processor 416 that receives and processes the ambient sound, and one or more speakers 414 that broadcast the attenuated sound for a wearer of hearing protection device 400. First hearing protection device 400 also includes a push-to-talk component 420 that includes an activator, such as a depressable button located on an exterior of the first hearing protector 400. However, as discussed herein, in some embodiments, a physical activator is not included. A voice-operated exchange may be used instead, for example.

First hearing protector 400 also includes a first communication component 424, which is illustrated in FIG. 4 as able to receive audio signals from first mobile communication component 430. However, it is expressly contemplated that first communication component 424 can also send digital communication signals to first mobile communication component 430. A second communication component, illustrated as first status communicator 422, is also present within first hearing protector 400. Status communicator 422 uses a low energy communication protocol to indicate to first mobile communication component 430 that the PTT component 420 has been activated or deactivated. In some embodiments, status communicator 422 periodically sends indications of the status of PTT component 420. However, particularly in embodiments where battery conservation is important, status communicator 422 may only send indications when a status of PTT component 420 changes, e.g. from deactivated to activated, or from activated to deactivated. Additionally, in some embodiments, status communicator 422 may only indicate when a PTT component 420 is activated.

First mobile communication component 430 includes first push-to-talk components 432, which may include a mobile application interface that receives the status communication from status communicator 422. Upon receiving a status indication indicating that a user of first hearing protector 400 wants the floor, first mobile communication component 430 may check the floor for availability and, if it is available, receive an audio signal from first communication component 424. First mobile communication component 430 then transmits the received audio signal from first hearing protector to second mobile communication component 480, and/or to third mobile communication component 530, which then transmit it, respectively, to second and third hearing protection devices 450, 500 using second PTT components 482, 532.

Similarly, if a user of second hearing protection device 450 or third hearing protection device 500 wants to use the PTT-over-cellular functionality, a status signal is sent from either second status communicator 472 or third status communicator 522 and received by second PTT component 482 or third PTT component 532. If the floor is open, then a user of either second device 450 or third device 500 can broadcast a signal using second cellular components 484 or third cellular components 534, which, respectively, receive the audio transmission from second communication component 474 or third communication component 524.

Second hearing protection device 450 and third hearing protection device 500, in some embodiments, have similar functionality to first hearing protection device 400, such that both include level dependent attenuation components 460, 510, which rely on receiving ambient sound through microphones 462, 512, and process the ambient sound using processors 466, 516, before broadcasting attenuated sound through speakers 464, 514. Both may also include other functionality 468, 518.

Status communicators 422, 472, and 522 all use a low energy communication protocol, such as BLE or another suitable low energy consuming communication protocol. Communication components 424, 474, and 524 may also use a low energy protocol, such as BLE, or may use conventional Bluetooth® protocols.

Mobile cellular phones 430, 480, and 530 may be any suitable ‘smart’ phone with the ability to connect to a cellular network, using cellular components 434, 484, and 534, and to pair with hearing protectors 400, 450, and 500. The pairing may use pairing technology separate from PPT components 432, 482, and 532, for example, such as traditional Bluetooth® pairing technology or fast pairing technology. While not illustrated in FIG. 4 for simplicity, mobile cellular devices 430, 480, and 530 may have other features, including user interfaces such as touchscreens, keyboards or buttons, as well as other features such as a battery and a processor. Similarly, hearing protection devices 400, 450 and 500 may also have additional functionality and components, including a rechargeable battery, cushioning, etc. Hearing protection devices 400, 450, and 500 may be any suitable hearing devices, including over-the-ear hearing protection devices, in-ear hearing protection devices, or dual-protection hearing devices such as those described in U.S. Provisional Patent Application 62/909989, filed on Oct. 3, 2019.

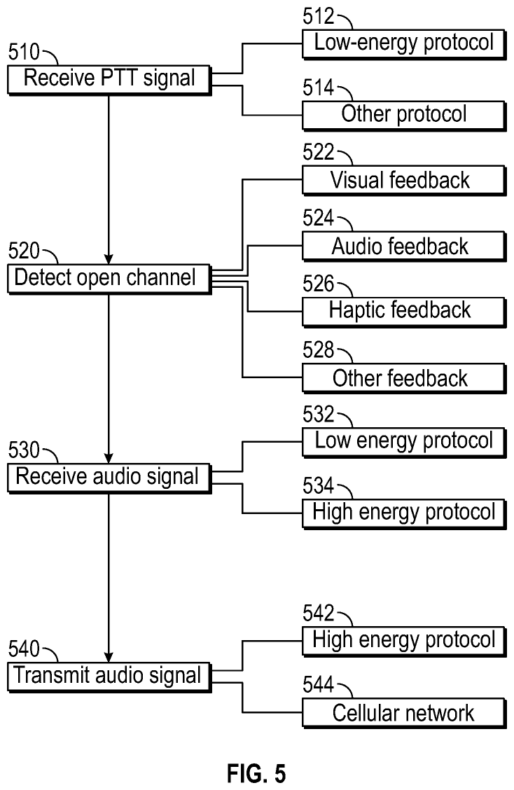

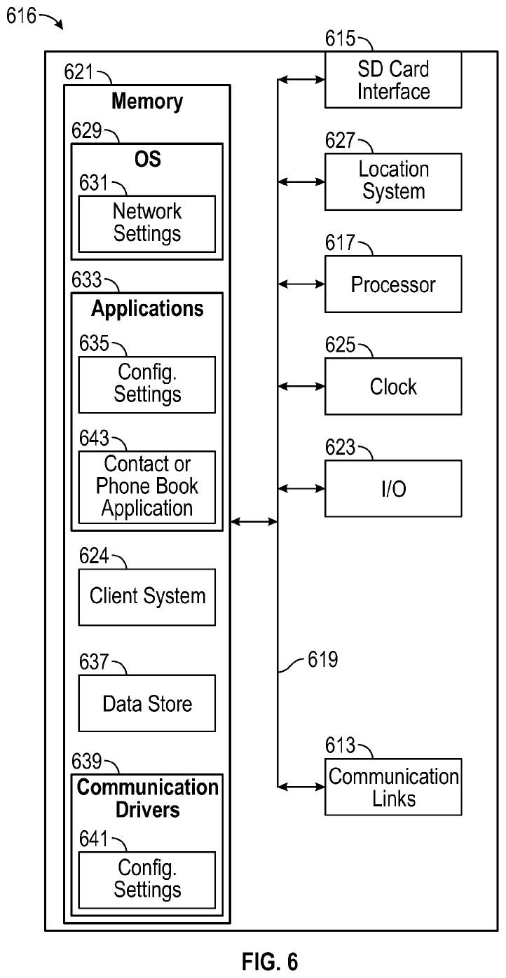

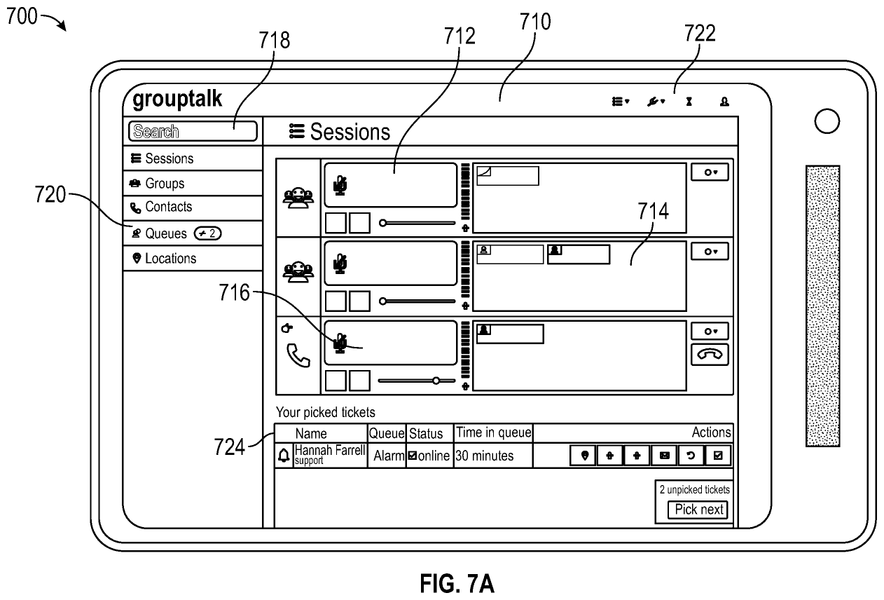

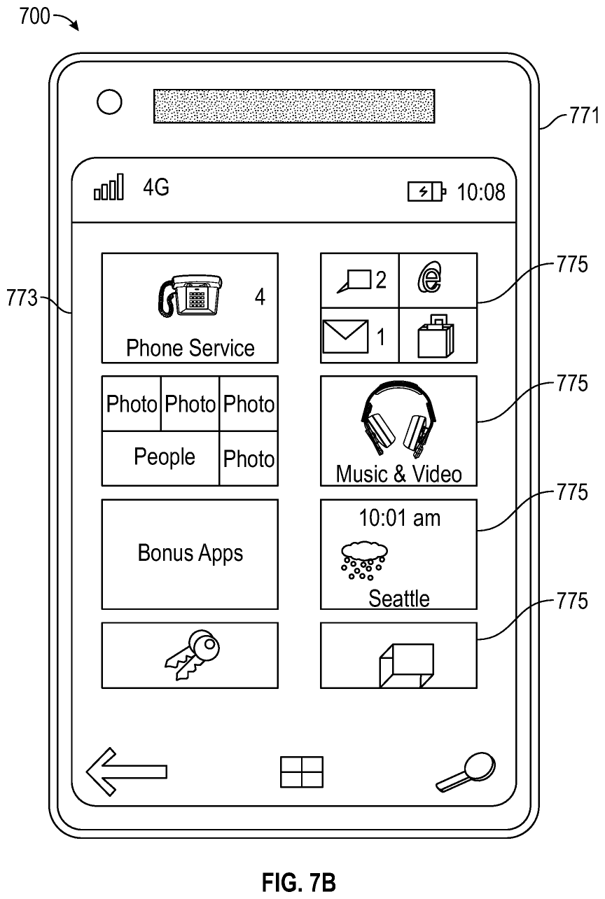

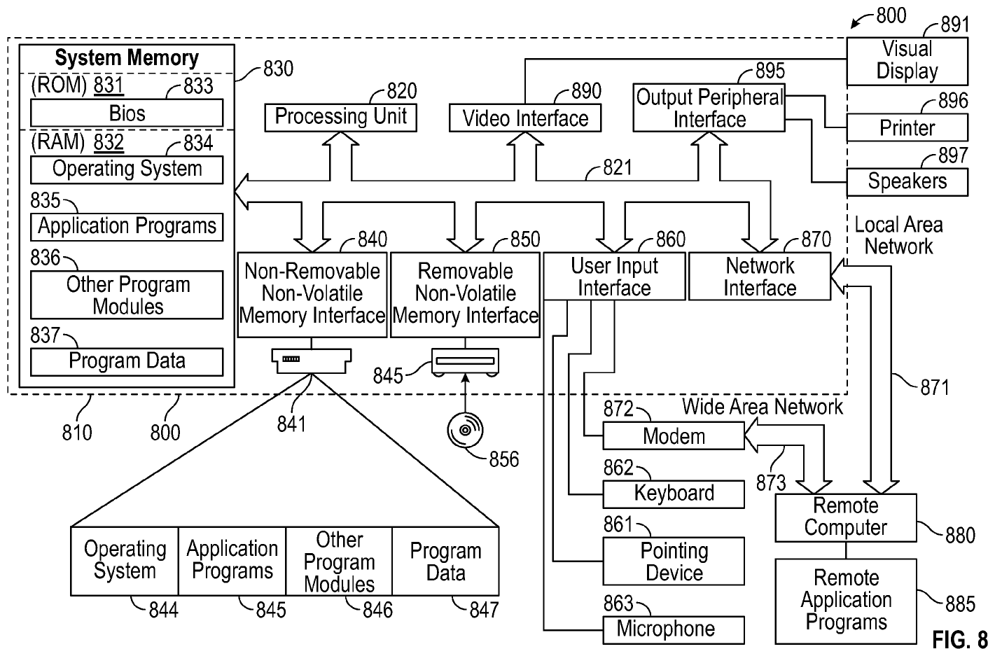

FIG. 5 illustrates a method of using push-to-talk over cellular enabled personal protective equipment. The personal protective equipment (PPE) can be, as illustrated in FIG. 2, over-the-ear headsets, in-ear plugs, or another suitable PPE. The method of 500 is illustrated from the perspective of a cellular phone paired with the PPE.

In block 510, a signal is received from the PPE indicating that the user of the PPE would like to use the PTT-over cellular functionality. The signal can be, in one embodiment, transmitted using a low-energy communication protocol, as indicated in block 512. The low energy protocol can be BLE, for example. However, other suitable protocols may also be used, as indicated in block 514. For example, in PPE where energy usage is less of a concern, traditional Bluetooth® may be used.

In block 520, an open channel for PTT communications is detected. The open channel may be detected, for example, by a mobile phone application checking whether an incoming PTT signal is being received from another device on the network. In some embodiments, an open channel will be present so long as there is network capacity and the mobile device is in range of the PPE. If an open channel is detected, feedback may be given to the wearer of the PPE. Additionally, negative feedback may be given if an open channel is not detected, so that the wearer is aware that they do not have an open floor. Feedback may be given by the mobile device itself, or a signal to give feedback may be sent from the mobile device to the PPE, which then delivers it to the user. In some use cases, the mobile phone may be in a pocket, backpack, toolkit, or otherwise not visible to a user. The type of feedback may be visual, as indicated in block 522, such as a flashing light indicating an open or closed signal. The type of feedback may also be audio, as indicated in block 524, such as a beep or voice cue that the floor is open or not. The type of feedback may also be haptic, such as either the mobile device or the PPE vibrating, as indicated in block 526. Other feedback options are also envisioned, as indicated in block 528. In block 530, an audio signal is received from the PPE. The audio signal may be a substantially simultaneous transmission from a microphone associated with the PPE, such that recipients of the PPT-over-cellular communication receive the transmission in substantially real-time. However, in some embodiments, because of the delay created by a cellular network in creating a mobile-to-mobile connection, the first message may be received at a delay. Subsequent messages may be received in substantially real-time, or may also be delayed, depending on the cellular network connection. Some processing of the captured audio may also occur, for example to heighten a quality of a wearer's speech and minimize ambient noise. However, in other embodiments the recorded speech is transmitted without processing. The audio signal may be received by a communication using a low-energy digital communication protocol, as indicated in block 532, or using a higher energy digital communication protocol, as indicated in block 534.

In block 540, the audio signal is transmitted from the cellular device to other cellular devices that are part of the PPT-over-cellular network. The audio signal may be transmitted using a high energy digital communication protocol, as indicated in block 542, if the receiving mobile device is within range. The audio signal can also be transmitted using a cellular network 544. A benefit of using the cellular network abilities of a mobile phone is that the audio signal can be transmitted further, without geographic limitation, and while maintaining audio quality. Other transmission methods are also possible. In some embodiments, a copy of the transmission may also be sent to a cloud storage, for example.

The audio signal may be transmitted to any cellular device that is in a selected network. For example, using an application interface, a user may be able to create a group of individuals that should receive a message, such as a group of workers in a given shift, or a group of workers at a given site. Additionally, a single recipient may be selected, or added to a group.

FIG. 6 illustrates an example mobile device that can be used in the embodiments shown in previous Figures. FIG. 6 is a simplified block diagram of one illustrative example of a handheld or mobile computing device that can be used as a user's or client's handheld device 212, 222, 232, 242 or 252, for example, in which the present system (or parts of it) can be deployed. For instance, a mobile device can be deployed in the operator compartment of computing device for use in generating, processing, or displaying the data.

FIG. 6 provides a general block diagram of the components of a mobile cellular device 616 that can run some components shown and described herein. Mobile cellular device 616 interacts with them or runs some and interacts with some. In the device 616, a communications link 613 is provided that allows the handheld device to communicate with other computing devices and under some embodiments provides a channel for receiving information automatically, such as by scanning. Examples of communications link 613 include allowing communication though one or more communication protocols, such as wireless services used to provide cellular access to a network, as well as protocols that provide local wireless connections to networks.

In other examples, applications can be received on a removable Secure Digital (SD) card that is connected to an interface 615. Interface 615 and communication links 613 communicate with a processor 617 (which can also embody a processor) along a bus 619 that is also connected to memory 621 and input/output (I/O) components 623, as well as clock 625 and location system 627.

I/O components 623, in one embodiment, are provided to facilitate input and output operations and the device 616 can include input components such as buttons, touch sensors, optical sensors, microphones, touch screens, proximity sensors, accelerometers, orientation sensors and output components such as a display device, a speaker, and or a printer port. Other I/O components 623 can be used as well.

Clock 625 illustratively comprises a real time clock component that outputs a time and date. It can also provide timing functions for processor 617.

Illustratively, location system 627 includes a component that outputs a current geographical location of device 616. This can include, for instance, a global positioning system (GPS) receiver, a LORAN system, a dead reckoning system, a cellular triangulation system, or other positioning system. It can also include, for example, mapping software or navigation software that generates desired maps, navigation routes and other geographic functions.

Memory 621 stores operating system 629, network settings 631, applications 633, application configuration settings 635, data store 637, communication drivers 639, and communication configuration settings 641. Memory 621 can include all types of tangible volatile and non-volatile computer-readable memory devices. It can also include computer storage media (described below). Memory 621 stores computer readable instructions that, when executed by processor 617, cause the processor to perform computer-implemented steps or functions according to the instructions. Processor 617 can be activated by other components to facilitate their functionality as well.

FIG. 7A illustrates an example device 700, with a user interface 710. User interface 710 may represent an example interface through which a user can interact with the PTT over cellular application on their cellular phone, tablet or other device 700. However, it is expressly contemplated that, in at least some embodiments, user does not need to interact with device 700 to initiate a call, and interacting with a PTT functionality on a headset will automatically result in device 700 initiating the PTT over cellular application on the phone.

User interface 710 may include one or more groups 712 that have been previously set up for easy communication. Groups may consist of one or more individuals, as indicated by group list 714. An active communication channel 716 may also be indicated on interface 710.

User interface may also have search features 718, and one or more menus such as on the left-hand side 720 or along the top 722 of an interface. Additionally, picked or unpicked tickets 724 may also be selectable.

FIG. 7B shows that the device can also be a smart phone 771. Smart phone 771 has a touch sensitive display 773 that displays icons or tiles or other user input mechanisms 775. Mechanisms 775 can be used by a user to run applications, make calls, perform data transfer operations, etc. In general, smart phone 771 is built on a mobile operating system and offers more advanced computing capability and connectivity than a feature phone. Note that other forms of the devices are possible.

FIG. 8 is one example of a computing environment in which elements of systems and methods described herein, or parts of them (for example), can be deployed. With reference to FIG. 8, an example system for implementing some embodiments includes a general-purpose computing device in the form of a computer 810. Components of computer 810 may include, but are not limited to, a processing unit 820 (which can comprise a processor), a system memory 830, and a system bus 821 that couples various system components including the system memory to the processing unit 820. The system bus 821 may be any of several types of bus structures including a memory bus or memory controller, a peripheral bus, and a local bus using any of a variety of bus architectures. Memory and programs described with respect to systems and methods described herein can be deployed in corresponding portions of FIG. 8.

Computer 810 typically includes a variety of computer readable media. Computer readable media can be any available media that can be accessed by computer 810 and includes both volatile/nonvolatile media and removable/non-removable media. By way of example, and not limitation, computer readable media may comprise computer storage media and communication media. Computer storage media is different from, and does not include, a modulated data signal or carrier wave. It includes hardware storage media including both volatile/nonvolatile and removable/non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computer 810. Communication media may embody computer readable instructions, data structures, program modules or other data in a transport mechanism and includes any information delivery media. The term “modulated data signal” means a signal that has one or more of its characteristics set or changed in such a manner as to encode information in the signal.

The system memory 830 includes computer storage media in the form of volatile and/or nonvolatile memory such as read only memory (ROM) 831 and random-access memory (RAM) 832. A basic input/output system 833 (BIOS) containing the basic routines that help to transfer information between elements within computer 810, such as during start-up, is typically stored in ROM 831. RAM 832 typically contains data and/or program modules that are immediately accessible to and/or presently being operated on by processing unit 820. By way of example, and not limitation, FIG. 8 illustrates operating system 834, application programs 835, other program modules 836, and program data 837.

The computer 810 may also include other removable/non-removable and volatile/nonvolatile computer storage media. By way of example only, FIG. 8 illustrates a hard disk drive 841 that reads from or writes to non-removable, nonvolatile magnetic media, nonvolatile magnetic disk 852, an optical disk drive 855, and nonvolatile optical disk 856. The hard disk drive 841 is typically connected to the system bus 821 through a non-removable memory interface such as interface 840, and optical disk drive 855 are typically connected to the system bus 821 by a removable memory interface, such as interface 850.

Alternatively, or in addition, the functionality described herein can be performed, at least in part, by one or more hardware logic components. For example, and without limitation, illustrative types of hardware logic components that can be used include Field-programmable Gate Arrays (FPGAs), Application-specific Integrated Circuits (e.g., ASICs), Application-specific Standard Products (e.g., ASSPs), System-on-a-chip systems (SOCs), Complex Programmable Logic Devices (CPLDs), etc.

The drives and their associated computer storage media discussed above and illustrated in FIG. 8, provide storage of computer readable instructions, data structures, program modules and other data for the computer 810. In FIG. 8, for example, hard disk drive 841 is illustrated as storing operating system 1844, application programs 845, other program modules 846, and program data 847. Note that these components can either be the same as or different from operating system 834, application programs 835, other program modules 836, and program data 837.

A user may enter commands and information into the computer 810 through input devices such as a keyboard 862, a microphone 863, and a pointing device 861, such as a mouse, trackball or touch pad. Other input devices (not shown) may include a joystick, game pad, satellite receiver, scanner, or the like. These and other input devices are often connected to the processing unit 820 through a user input interface 860 that is coupled to the system bus but may be connected by other interface and bus structures. A visual display 891 or other type of display device is also connected to the system bus 821 via an interface, such as a video interface 890. In addition to the monitor, computers may also include other peripheral output devices such as speakers 897 and printer 896, which may be connected through an output peripheral interface 895.

The computer 810 is operated in a networked environment using logical connections, such as a Local Area Network (LAN) or Wide Area Network (WAN) to one or more remote computers, such as a remote computer 880.

When used in a LAN networking environment, the computer 810 is connected to the LAN 871 through a network interface or adapter 870. When used in a WAN networking environment, the computer 810 typically includes a modem 872 or other means for establishing communications over the WAN 873, such as the Internet. In a networked environment, program modules may be stored in a remote memory storage device. FIG. 8 illustrates, for example, that remote application programs 885 can reside on remote computer 880.

A hearing protection device is presented that includes a first hearing protection unit and a second hearing protection unit. Each of the first and second hearing protection units has a microphone that receives ambient sound, a processor that provides level dependent attenuation of the ambient sound, and a speaker that broadcasts the sound into a user's ear. The hearing protection device also has a first communication unit, which broadcasts a digital push-to-talk signal over a first communication protocol. The first communication protocol is a low energy communication protocol. The hearing protection device also includes a second communication unit, which broadcasts a digital communication signal over a second communication protocol. The hearing protection device also includes a processor that facilitates pairing of the hearing protection device with a mobile device that receives the push-to-talk signal and, based on the push-to-talk signal, receives the digital communication signal and transmits the digital communication signal over a cellular network, such as 2G, 3G, 4G, 5G, CDMA, TDMA, FDMA or any other suitable data provider that is compatible with a given mobile cellular device.

The hearing protection device may be implemented such that the first communication protocol uses a 2.44-2.4835 GHz ISM band. The hearing protection device may be implemented such that the first communication protocol has forty 2 MHz channels.

The hearing protection device may be implemented such that the second communication protocol uses a 2.44-2.4835 GHz ISM band.

The hearing protection device may be implemented such that the first and second hearing protection units are over-the-ear muffs.

The hearing protection device may be implemented such that the first and second over-the-ear muffs are connected by a headband.

The hearing protection device may be implemented such that the first and second over-the-ear muffs are wired together.

The hearing protection device may be implemented such that the first and second communication units are both in the first over-the-ear muffs.

The hearing protection device may be implemented such that the first over-the-ear muff includes a rechargeable battery.

The hearing protection device may be implemented such that the rechargeable battery provides power to both the first and second over-the-ear muffs.

The hearing protection unit may be implemented such that the first and second hearing protection units include in-ear units.

The hearing protection unit may be implemented such that the microphone is part of a portion of the in-ear unit extending inside a user's ear canal.

The hearing protection unit may be implemented such that each of the first and second hearing protection units have a rechargeable battery.

A method of sending communication between personal protective hearing devices using push-to-talk over cellular communication. The method includes receiving, on a first mobile cellular device, a signal from a first personal protective hearing device on a first communication protocol, the signal comprising a request to take control of a cellular network connection. The first communication protocol is a low energy protocol. The method also includes detecting, using the first mobile cellular device, that the cellular network connection between the first mobile cellular device and a second mobile cellular device is open. The method also includes receiving, on the first mobile cellular device, from the first personal protective hearing device, a digital audio communication from the first personal protective hearing device over a second communication protocol. The method also includes transmitting the digital audio communication from the first mobile device to a second mobile device using a cellular network connection.

The method may be implemented such that it also includes providing feedback, using the first mobile device, that the cellular network connection is available for a PPT-over cellular communication.

The method may be implemented such that the provided feedback is a feedback signal, provided from the first mobile device to the first personal protective hearing device. The feedback signal includes a command for the personal protective hearing device to provide feedback.

The method may be implemented such that the provided feedback is audio, visual or other feedback.

The method may be implemented such that it also includes transmitting the digital audio communication from the first mobile device to a third mobile device.

The method may be implemented such that transmitting the digital audio communication from the first mobile device occurs substantially simultaneously to the second and third mobile devices.

The method may be implemented such that it includes transmitting the digital audio communication from the second mobile device to a second personal protective hearing device, using the second communication protocol.

The method may be implemented such that detecting that the cellular network connection is open includes detecting no incoming transmissions from the second mobile device or sending a signal verifying that a second personal protective hearing device has not activated a PPT-over cellular functionality.

The method may be implemented such that the only communication channel between the first and second personal protective hearing devices is through the first and second mobile devices.

The method may be implemented such that the first and second mobile devices are cellular devices. Receiving the signal, detecting the open cellular network connection, receiving the audio signal and transmitting the audio signal may be facilitated by a mobile application installed on the cellular devices.

The method may be implemented such that the first communication protocol is different from the second communication protocol.

The method may be implemented such that the first communication protocol uses a 2.44-2.4835 GHz ISM band.

The method may be implemented such that the first communication protocol has forty 2 MHz channels.

The method may be implemented such that the second communication protocol uses a 2.44-2.4835 GHz ISM band.

The method may be implemented such that the first and second hearing protection units are over-the-ear muffs.

The method may be implemented such that the first and second over-the-ear muffs are connected by a headband.

The method may be implemented such that the first and second over-the-ear muffs are wired together.

The method may be implemented such that the first and second communication units are both in the first over-the-ear muffs.

The method may be implemented such that the first over-the-ear muff includes a rechargeable battery.

The method may be implemented such that the rechargeable battery provides power to both the first and second over-the-ear muffs.

The method may be implemented such that the first and second hearing protection units include in-ear units.

The method may be implemented such that the microphone is part of a portion of the in-ear unit extending inside a user's ear canal.

The method may be implemented such that each of the first and second hearing protection units have a rechargeable battery.

Computer-implemented instructions are stored in a memory on a smartphone that cause the smartphone to: pair the smartphone to a first personal protective hearing device. Pairing includes the smartphone and the first personal protective hearing device paired for communication over a first communication protocol and a second communication protocol. The instructions also cause the smartphone to receive a digital communication signal from the personal protective device, over the first communication protocol. The digital communication signal includes a command to initiate a PTT-over cellular communication link between the first personal protective hearing device and a second personal protective hearing device. The instructions also cause the smartphone to receive a digital audio communication signal from the first personal protective hearing device and transmit the digital audio communication to a second smartphone over a cellular network. The first communication protocol is a low energy communication protocol.

The computer implemented instructions may be implemented such that the first communication protocol is different from the second communication protocol.

The computer implemented instructions may be implemented such that the first communication protocol uses a 2.44-2.4835 GHz ISM band.

The computer implemented instructions may be implemented such that the first communication protocol has forty 2 MHz channels.

The computer implemented instructions may be implemented such that the second communication protocol uses a 2.44-2.4835 GHz ISM band.

The computer implemented instructions may be implemented such that the first and second hearing protection units are over-the-ear muffs.

The computer implemented instructions may be implemented such that the first and second over-the-ear muffs are connected by a headband.

The computer implemented instructions may be implemented such that the first and second over-the-ear muffs are wired together.

The computer implemented instructions may be implemented such that the first and second communication units are both in the first over-the-ear muffs.

The computer implemented instructions may be implemented such that the first over-the-ear muff includes a rechargeable battery.

The computer implemented instructions may be implemented such that the rechargeable battery provides power to both the first and second over-the-ear muffs.

The computer implemented instructions may be implemented such that the first and second hearing protection units include in-ear units.

The computer implemented instructions may be implemented such that the microphone is part of a portion of the in-ear unit extending inside a user's ear canal.

The computer implemented instructions may be implemented such that each of the first and second hearing protection units have a rechargeable battery.

A network of personal protective equipment includes a first personal protection device paired to a first mobile cellular device and a second personal protection device paired to a second mobile cellular device. The first personal protection device and the second personal protection device are configured to communicate using a PTT-over cellular communication network.

The network may be implemented such that using a PTT-over cellular communication network further includes the first personal protection device transmitting a request to broadcast over the PTT-over cellular communication network to the first mobile cellular device using a first communication protocol, the first personal protection device transmitting a digital audio signal to broadcast over the PTT-over cellular communication network to the first mobile cellular device using a second communication protocol, and the first mobile cellular device transmitting the digital audio signal to the second mobile cellular device using a cellular network connection and the second mobile cellular device transmitting the digital audio signal to the second personal protection device.

The network may be implemented such that the first communication protocol is a low energy communication protocol.

The network may be implemented such that the second communication protocol is a low energy communication protocol.

The network may be implemented such that the first communication protocol is different from the second communication protocol.

The network may be implemented such that the first communication protocol uses a 2.44-2.4835 GHz ISM band.

The network may be implemented such that the first communication protocol has forty 2 MHz channels.

The network may be implemented such that the second communication protocol uses a 2.44-2.4835 GHz ISM band.

The network may be implemented such that the first and second hearing protection units are over-the-ear muffs.

The network may be implemented such that the first and second over-the-ear muffs are connected by a headband.

The network may be implemented such that the first and second over-the-ear muffs are wired together.

The network may be implemented such that the first and second communication units are both in the first over-the-ear muffs.

The network may be implemented such that the first over-the-ear muff includes a rechargeable battery.

The network may be implemented such that the rechargeable battery provides power to both the first and second over-the-ear muffs.

The network may be implemented such that the first and second hearing protection units include in-ear units.

The network may be implemented such that the microphone is part of a portion of the in-ear unit extending inside a user's ear canal.

The network may be implemented such that each of the first and second hearing protection units have a rechargeable battery.

1. A hearing protection device comprising:
a first hearing protection unit and a second hearing protection unit, wherein each of the first and second hearing protection units comprise:
a microphone that receives ambient sound;
a processor that provides level dependent attenuation of the ambient sound; and
a speaker that broadcasts the sound into a user's ear;
a first communication unit, which broadcasts a digital push-to-talk signal over a first communication protocol, wherein the first communication protocol is a low energy communication protocol;
a second communication unit, which broadcasts a digital communication signal over a second communication protocol; and
a processor that facilitates pairing of the hearing protection device with a mobile device that receives the push-to-talk signal and, based on the push-to-talk signal, receives the digital communication signal and transmits the digital communication signal over a cellular network.

2. The hearing protection device of claim 1, wherein the first communication protocol or the second communication protocol uses a 2.44-2.4835 GHz ISM band.

3. The hearing protection device of claim 2, wherein the first communication protocol has forty 2 MHz channels.

4-7. (canceled)

8. The hearing protection device of claim 1, wherein the first and second communication units are both in a pair of over-the-ear muffs.

9. The hearing protection device of claim 8, wherein the first over-the-ear muff comprises a rechargeable battery and wherein the rechargeable battery provides power to both the first and second over-the-ear muffs.

10. (canceled)

11. The hearing protection unit of claim 1, wherein the first and second hearing protection units comprise in-ear units.

12. The hearing protection unit of claim 1, wherein the microphone is part of a portion of the in-ear unit extending inside a user's ear canal.

13. The hearing protection unit of claim 11, wherein each of the first and second hearing protection units have a rechargeable battery.

14. A method of sending communication between personal protective hearing devices using push-to-talk over cellular communication, the method comprising:
receiving, on a first mobile cellular device, a signal from a first personal protective hearing device on a first communication protocol, the signal comprising a request to take control of a cellular network connection, wherein the first communication protocol is a low energy protocol;
detecting, using the first mobile cellular device, that the cellular network connection between the first mobile cellular device and a second mobile cellular device is open;
receiving, on the first mobile cellular device, from the first personal protective hearing device, a digital audio communication from the first personal protective hearing device over a second communication protocol; and
transmitting the digital audio communication from the first mobile device to a second mobile device using a cellular network connection.

15. The method of claim 14, and further comprising: providing feedback, using the first mobile device, that the cellular network connection is available for a PPT-over cellular communication.

16. The method of claim 15, wherein the provided feedback is a feedback signal, provided from the first mobile device to the first personal protective hearing device, wherein the feedback signal comprises a command for the personal protective hearing device to provide feedback and, wherein the provided feedback is audio, visual or other feedback.

17. (canceled)

18. The method of claim 14, and further comprising:
transmitting the digital audio communication from the first mobile device to a third mobile device, and wherein transmitting the digital audio communication from the first mobile device occurs substantially simultaneously to the second and third mobile devices.

19. (canceled)

20. The method of claim 14, and further comprising:
transmitting the digital audio communication from the second mobile device to a second personal protective hearing device, using the second communication protocol.

21. The method of claim 14, wherein detecting that the cellular network connection is open comprises:
detecting no incoming transmissions from the second mobile device; or
sending a signal verifying that a second personal protective hearing device has not activated a PPT-over cellular functionality.

22. The method of claim 21, wherein the only communication channel between the first and second personal protective hearing devices is through the first and second mobile devices.

23. The method of claim 14, wherein the first and second mobile devices are cellular devices, and wherein receiving the signal, detecting the open cellular network connection, receiving the audio signal and transmitting the audio signal are facilitated by a processing unit of the cellular devices.

24. The method of claim 14, wherein the first communication protocol is different from the second communication protocol.

25-27. (canceled)

28. The method of claim 14, wherein the first and second hearing protection units are over-the-ear muffs.

29-33. (canceled)

34. The method of claim 14, wherein the first and second hearing protection units comprise in-ear units and wherein the microphone is part of a portion of the in-ear unit extending inside a user's ear canal.

35. (canceled)

36. (canceled)

37. Computer-implemented instructions stored in a memory on a smartphone, the instructions causing the smartphone to:
pair the smartphone to a first personal protective hearing device, wherein pairing comprises the smartphone and the first personal protective hearing device paired for communication over a first communication protocol and a second communication protocol;
receive a digital communication signal from the personal protective device, over the first communication protocol, wherein the digital communication signal includes a command to initiate a PTT-over cellular communication link between the first personal protective hearing device and a second personal protective hearing device;
receive a digital audio communication signal from the first personal protective hearing device; and
transmit the digital audio communication to a second smartphone over a cellular network; and
wherein the first communication protocol is a low energy communication protocol.
